(19) United States
(12) Patent Application Publication
(10) Pub. No.: US 2021/0054106 A1
(43) Pub. Date: Feb. 25, 2021

(54) OMNIPHILIC NANO-VESICLES BASED ON MODIFIED POLYSACCHARIDES FOR DELIVERY OF ACTIVE AGENTS

(71) Applicant: THE STATE OF ISRAEL, MINISTRY OF AGRICULTURE & RURAL DEVELOPMENT, AGRICULTURAL RESEARCH ORGANIZATION, Rishon Lezion (IL)

(72) Inventors: Elena Poverenov, Nes Ziona (IL); Roi Rutenberg, Tel Aviv (IL); Yael Cohen, Ramat-Gan (IL)

(21) Appl. No.: 16/982,827

(22) PCT Filed: Mar. 24, 2019
(86) PCT No.: PCT/IL2019/050329

§ 371 Date: Sep. 21, 2020

(30) Foreign Application Priority Data
Mar. 25, 2018 (IL) 258325

(51) Int. Cl. C08B 37/08; C08L 5/08; A01N 25/26; A61K 9/51; C05G 5/30

(52) U.S. Cl. CPC C08B 37/003; C08L 5/08; B82Y 5/00; A61K 9/5161; C05G 5/37; A01N 25/26

(57) ABSTRACT

The present invention relates to modified polysaccharide-based self-assembled aggregates capable to encapsulate and deliver active agents into and between media of different hydrophilicity/hydrophobicity.

### FIELD OF THE INVENTION

The present invention relates to modified polysaccharide-based aggregates capable to deliver active agents into and between media of different hydrophilicity/hydrophobicity. Particularly, the invention relates to aggregates which spontaneously self-assemble and are capable to encapsulate various active agents (both hydrophobic and hydrophilic) and deliver them into variable environments, including environments in which the active agents are sparely soluble.

### BACKGROUND OF THE INVENTION

Biopolymer-based nanostructures have been widely studied in recent years for their use as advanced delivery systems. Selecting nature-sourced polymers as a design starting point is advantageous in yielding new materials with desired biocompatibility for controlled release and targeted transport of active agents. Such biopolymer-based delivery systems can potentially contribute to many fields such as medicine, pharmacology, food, cosmetics, and more. Polysaccharides are of particular interest as biopolymer candidates due to their low price, availability, and defined chemical structures, examples being polysaccharides such as carrageenan, alginate, chitin, chitosan, cellulose and its derivatives and others. However, these materials usually lack amphiphilic character, important for enveloping both hydrophobic and hydrophilic loads of high applicative interest. It is therefore an object of the invention to provide a biopolymer system for delivering active agents based on modified polysaccharides which would avoid the known drawbacks. EP 1001790 describes cationic polymers having hydrophobic groups, such as hydrophobized polysaccharides, for lubricating and separating tissues and biological membranes, preferably with classical surface-active agents such as phospholipids. WO 2017/014655 describes nanocapsules for delivering lipophilic compound without employing classical low-molecular weight surfactants, while possibly using chitosan re-acetylated on the free amino groups.

It is an object of this invention to provide a delivery system based on polysaccharides exhibiting amphiphilic characteristics due to modifying moieties that include hydrophobic side chains.

It is a further object of this invention to provide modified polysaccharides which self-assemble into stable sub-micron aggregates that can deliver both hydrophobic and hydrophilic agents into or through varied environments of different hydrophilicity/hydrophobicity.

It is still another object of this invention to provide modified polysaccharides which self-assemble into stable sub-micron aggregates that can deliver hydrophobic agents into or through varied environments of different hydrophilicity/hydrophobicity.

It is still further object of this invention to provide modified polysaccharides which self-assemble into stable sub-micron aggregates that can deliver hydrophilic agents into or through varied environments of different hydrophilicity/hydrophobicity.

This invention aims at providing a omniphilic polysaccharide-based system (OPN) capable of delivering active agents between hydrophilic and lipophilic environments.

This invention also aims at providing a polysaccharide delivery system enabling to load an active agent in a lipophilic environment and release said agent in a hydrophilic environment.

It is also an object of this invention to provide a simple system for testing the ability of polysaccharide aggregates to deliver agents between hydrophilic and lipophilic environments.

Other objects and advantages of present invention will appear as the description proceeds.

### SUMMARY OF THE INVENTION

This invention provides amphiphilic aggregates comprising an hydrophilic polysaccharide modified by covalently linked moieties that contain aliphatic chains comprising between 2 and 26 carbon atoms and degree of substitution with aliphatic moieties constitutes between 0.5 and 70%, said modified polymer exhibiting a critical aggregate concentration (CAC) of between 0.00005 and 2.0 mg/ml. Said moiety is preferably linked to the polysaccharide via a bond selected from the group consisting of amine bond, amide bond, N-imine bond, ester bond, and ether bond. In an important aspect, the invention provides aggregates based on a modified polysaccharide which complies with the Rhodamine test (defined below).

The Rhodamine delivery test is employed by the present invention to select suitable modified amphiphilic polysaccharide-based aggregates. The Rhodamine test consists of the following steps:
i) providing 0.5 mL of aqueous solution of a modified amphiphilic polysaccharide into 10 mL of a hydrophobic organic solvent such as xylene or oil such as sunflower oil and heating of the mixture at 105° C. for 20 min to remove water traces. The obtained concentration of the modified amphiphilic polysaccharide in the new solvent has to be at least 3.5 times higher than its CAC (critical aggregation concentration). For example, 10 mg of chitosan(CS)-8 modified amphiphilic polysaccharide in 10 ml of xylene (or oil) were used in this test. The amount of the modified polysaccharide used in this test could be different (depending on its CAC), however it should be at least was 3.5 times larger than its CAC.
   Then, the mixture was eventually stirred or shaken at an ambient temperature for maximum 2 hrs;
ii) dispersing 3 mg of Rhodamine B in the 10 ml of xylene (or oil) containing the tested modified amphiphilic polysaccharide and stirring at ambient temperature for maximum 2 hrs. Then, the color of the hydrophobic solvent changes from colorless to red/pink-red/violet-red/purple-red (depends on solvent) or orange-red (in case of oil that was originally yellow color);
iii) adding 1 volume of water (10 ml) to the hydrophobic mixture and agitating with a vortex at ambient temperature for 1 min, then stirring for 5 minutes and then waiting for additional 5 minutes till the phases separate;
iv) visually evaluating the color of the water phase;
   if the color of the water phase changes colorless to characteristic Rhodamin B color (pink-violet) in step iv, then the modified amphiphilic polysaccharide complies with the Rhodamine test and the material can be employed in a delivery system according to the invention.

For brevity, the suitable material will be referred to hereinafter as “complying with the Rhodamine test”.

The invention relates to a delivery system comprising an amphiphilic aggregate essentially consisting of a modified polysaccharide complying with the Rhodamine test. The delivery system of the invention is based on a polysaccharide modified by covalently linked moieties that contain aliphatic chains comprising between 2 and 26 carbon atoms linked via a bond selected from the group consisting of amine bond, amide bond, N-imine bond, ester bond, and ether bond. Said degree of substitution with aliphatic moieties constitutes between 0.5 and 70%. Said modified polymer exhibits a critical aggregate concentration (CAC) of between 0.00005 and 2 mg/ml. The delivery system of the invention is suitable for delivering hydrophobic and hydrophilic agents into or between hydrophilic and lipophilic environments.

### BRIEF DESCRIPTION OF THE DRAWINGS

The above and other characteristics and advantages of the invention will be more readily apparent through the following examples, and with reference to the appended drawings, wherein:

FIG. 1. shows fluorescence intensity changes in 1 μM concentration of pyrene (1A), Rhodamine B (1B), and curcumin (1C) when introduced to CS-4-OPNs, CS-8-OPNs or CS-12-OPNs (CS-4, CS-8 or CS-12, respectively) aggregate aqueous solutions.

FIG. 2. Shows CLSM images of Rhodamine B (RB), pyrene (pyr), and both probes simultaneously encapsulated by non-stimulated (NS)CS-12-OPNs (CS-12) aggregates, or after application of acidic stimulus (AS).

FIG. 3. shows spectrofluorometric scans and color change of xylene upon solubilization of Rhodamine B (RB) in the presence of amphiphilic modified polysaccharide-based aggregates, CS-OPNs, or lack of Rhodamine B solubilization in the absence of amphiphilic modified polysaccharide-based aggregates, CS-OPNs.

FIG. 4. shows CLSM images of sunflower oil upon solubilization of Rhodamine B (RB) in the presence of amphiphilic modified polysaccharide-based aggregates, CS-OPNs (+agg) or lack of Rhodamine B solubilization in the absence of amphiphilic modified polysaccharide-based aggregates, CS-OPNs (− agg).

FIG. 5. shows percent of fungal inoculation in wheat grains for 1-Octanol (1-Oct), Eugenol (Eug), Thymol (Thy), Oreganum Oil (Oreo) and Tea Tree Oil (TTO) as directly added bioactive agents, and for Untreated (UN), Acidic Stimulus (AS) alone, CS-6-OPNs (OPN-6) alone, Hexanal (Hex) and CS-6-OPNs with Acidic Stimulus (OPN-6+AS), as controls after 10 days of exposure time.

FIG. 6. shows percent of fungal inoculation in wheat grains over time (T) when treated with an CS-6-OPNs (CS-6) formulation with encapsulated tea tree oil.

FIG. 7. shows light microscopy enlargements of wheat grains with heavy (7A), partial (7B) and minimal (7C) fungal inoculations.

FIG. 8. shows SEM-EDS microscopic images of Zn, Mn, SO₄ and CI ions in plant leaves measured after 60 minutes of treatment with 100 ppm ZnSO₄ (8A), MnSO₄ (8B), ZnCl₂ (8C) or MnCl₂ (8D) unencapsulated (U) or encapsulated in unmodified CS-OPNs (CS), CSreduced-4-OPNs (C4), CSreduced-8-OPNs (C8) or CSreduced-12-OPNs (C12). The bar indicates 500 μm.

FIG. 9. shows roots of Maize plants treated with ZnSO₄ unencapsulated or encapsulated in CMC-OPNs (“CMC”) or CSreduced-OPNs (“Chitosan”) having 0, 4, 8 or 12 carbon alkyl chain (CS, C4, C8 and C12, respectively).

FIG. 10. shows an illustration of Franz diffusion cell experiment for CS-OPNs (OPNs) loaded with Rhodamine B (RB) in sunflower oil (10A). Passage across a lipophilic membrane was recorded using a spectrofluorometer as sampled from the aqueous phase. Successful stability and cross-phase transport over time (T) of RB encapsulated in CS-4-OPNs, CS-8-OPNs or CS-12-OPNs (CS-4, CS-8 or CS-12, respectively) compared to unencapsulated RB (U) is shown in FIG. 10B.

### DETAILED DESCRIPTION OF THE INVENTION

It has now been found that polysaccharides modified by covalent linkage of moieties that contain aliphatic chains efficiently solubilize hydrophilic agents in lipophilic environments and hydrophobic agents in aqueous environment, and further that said modified polysaccharides efficiently deliver said agents between two environments of different hydrophobicity. Moreover, as an option, said delivery system can be dissembled by external factor, for example when the polysaccharide modification involves dynamic covalent bond. In one embodiment, decreasing pH can disassemble aliphatic chains linked to chitosan by an N-alkylimine linkage.

In one embodiment, the invention provides a delivery system based on modified chitosan, in which an aliphatic aldehyde reacted with at least a part of free amine groups, forming Schiff base and covalently binding the aliphatic moiety to the carbohydrate oligomer or polymer. The resulting modified chitosan exhibits amphiphilic character, allowing the molecules to self-assemble in either hydrophilic or lipophilic environment into stable sub-micron aggregates that can encapsulate both hydrophilic and hydrophobic guests. It has been found that these aggregates, after immersing in a lipophilic environment and contacting with a hydrophilic agent, encapsulate said agent in said lipophilic milieu. This provides a controlled-release delivery system according to one embodiment of the invention. The delivery system has tremendous flexibility, ease of use, and a large applicable potential, for example that allows delivery of various active agents such as drugs, food supplements, plant nutrients and biostimulants, cosmetic agents, ect.

The amphiphilic aggregates based on modified polysaccharides are prepared by covalent modifications of hydrophilic polysaccharides, such as cellulose derivatives, pectins, alginates and chitosans, with alkyl chain-containing modifying moieties. For example, aliphatic chains are covalently linked to amino groups or hydroxyl groups or carboxyl groups of hydrophilic polymers, such as chitosan or carboxymethyl cellulose (CMC), the aliphatic chains being provided in aldehydes, anhydrides, amines, acyl chlorides, and other known reagents. In a preferred embodiment, aliphatic aldehydes are coupled to the free amino groups of polysaccharide chitosan.

In general, the present invention provides three types of amphiphilic nano-vesicles. In one aspect, an N-alkylimine bond is created, providing a breakable (dynamic) covalent bond. In one embodiment, the nano-vesicle contains pH responsive N-imine dynamic covalent bonds, and is designated herein “chitosan-based omniphilic polysaccharide nanopackets” or “CS-OPN”.

In a second aspect, a stable amine bond is formed when desired, by reducing the N-alkylimine bond by known methods. In one embodiment, the nano-vesicle contains a stable secondary amine bond, forming a chitosan-based OPN in which N-imine bond is reduced. This type of nano-vesicle is designated herein as “CSreduced-OPN”.

In a third aspect, a stable amide bond is formed by EDC-NHS (1-Ethyl-3-(3-dimethylaminopropyl)carbodiimide-N-Hydroxysuccinimide) coupling. In one embodiment, the functional group carbodiimide is a dehydration agent, and with the additive NHS that activates the acid, it is a suitable reaction for coupling the carboxymethyl cellulose (CMC) acid with aliphatic amines. The obtained nano-vesicle is termed herein “CMC-OPN”.

In a series of experiments, a series of aliphatic aldehydes, between 4 and 12 carbon atoms, were reacted with chitosan, providing dynamic (detachable)N-alkylimine bond (CS-OPNs). In other experiments, a series of aliphatic aldehydes, between 4 and 12 carbon atoms, were reacted with chitosan to obtain N-alkylimines that were then reduced to provide a stable N-alkylamine bond (CSreduced-OPNs). In additional experiments, a series of aliphatic amines, between 4 and 12 carbon atoms, were reacted with carboxymethyl cellulose, to provide a stable alkylamide bond (CMC-OPNs).

These modified polysaccharides combined hydrophobicity of the aliphatic chains with hydrophilicity of the sugar units, resulting in their self-assembly into stable submicron aggregates of a relatively narrow size distribution; usually, the aggregates have essentially spherical shape. The active payload encapsulation was demonstrated for both hydrophilic agents, such as Rhodamine B, and hydrophobic agents, such as pyrene or curcumin, and other model agents. These nano-vesicles have great flexibility and allow to regulate their composition to be adapted for various payloads and environments. Their flexibility also allows them to structurally adapt to a hydrophilic and hydrophobic microenvironment.

As an option, delivery system can be dissembled by external factor when the polysaccharide modification involves dynamic covalent bond. In one embodiment, chitosan modified by an N-alkylimine linkage can deconstruct upon introducing an external pH trigger. An acidic trigger cleaves the N-alkylimine linkage and reverts the nanoaggregates back to their original un-aggregated state, uncoupling the aldehydes and releasing the payload. In one aspect of the invention, the aldehyde for the delivery system according to the invention is selected for coupling to the amino group according to its synthetic compatibility and desired hydrophobicity; in another aspect of the invention, the aldehyde is selected according to its biological properties—possibly being an active agent by itself or contributing to the formation of an active agent. Particularly, the uncoupled and released aldehyde may act as an antioxidant, as an antimicrobial or antifungal or antibacterial agent, potentially more active as a free molecule than as a coupled chain. The dynamic bond strategy thus ensures a temporal inertness of components until the opportune moment, and provides a delivery system with advanced, multi-level biological activities. In such a case, an active material may be present either in said payload or as a component comprised in the structure of the modified amphiphilic polysaccharide.

The biopolymer-based aggregates of the invention demonstrate remarkable flexibility under various conditions and ability to assimilate in media of different polarities. Due to their ability to rearrange, they have applicative potential in an aqueous environment, in a lipophilic environment, and in through-phases delivery.

In an important aspect of the invention, amphiphilic aggregates comprising a chemically modified hydrophilic polymer are provided for delivering active agents between hydrophilic and lipophilic environments. Particularly, a modified polysaccharide is provided to deliver a low-molecular agent from the aqueous phase to an oil phase, and from the oil phase to an aqueous phase. Low-molecular agents in this context having a molecular weight of less than 5000, such as less than 2000, for example less than 1000. Examples of such low-molecular active agents comprise drugs, antimicrobials, nutraceutics, vitamins, biostimulants, plant nutrients, etc. The amphiphilic modified polysaccharide usually self-assembles in the oily phase or in aqueous phase to form aggregates or vesicles. A model agent to test the ability is Rhodamine B, whose delivery is indicated in this context by the appearance of its typical color in the liquid phase.

The Rhodamine delivery test is employed by the present invention to select suitable modified amphiphilic polysaccharides. As aforementioned, the Rhodamine test consists of the following steps:
i) providing 0.5 mL of aqueous solution of a modified amphiphilic polysaccharide into 10 mL of a hydrophobic organic solvent such as xylene or oil such as sunflower oil and heating of the mixture at 105° C. for 20 min to remove water traces. The obtained concentration of the modified amphiphilic polysaccharide in the new solvent has to be at least 3.5 times higher than its CAC (critical aggregation concentration). For example, 10 mg of CS-8 modified amphiphilic polysaccharide in 10 ml of xylene (or oil) were used in this test. The amount of the modified polysaccharide used in this test could be different (depending on its CAC), however it should be at least was 3.5 times larger than its CAC;
   then, the mixture was eventually stirred or shaken at an ambient temperature for maximum 2 hrs;
ii) dispersing 3 mg of Rhodamine B in the 10 ml of xylene (or oil) containing the tested modified amphiphilic polysaccharide and stirring at ambient temperature for maximum 2 hrs. Then, the color of the hydrophobic solvent changes from colorless to red/pink-red/violet-red/purple-red (depends on solvent) or orange-red (in case of oil that was originally yellow color);
iii) adding 1 volume of water (10 ml) to the hydrophobic mixture and agitating with a vortex at ambient temperature for 1 min, then stirring for 5 minutes and then waiting for additional 5 minutes till the phases separate;
iv) visually evaluating the color of the water phase;
   if the color of the water phase changes colorless to characteristic Rhodamin B color (pink-violet) in step iv, then the modified amphiphilic polysaccharide complies with the Rhodamine test and the material can be employed in a delivery system according to the invention.

In a preferred embodiment of the invention, provided is a system for delivering active agents by means of polysaccharide nano-vesicles, which bind or encapsulate said agents and assist in temporary protecting said agents and/or transferring them. In a particularly preferred embodiment, said polysaccharide comprises chitosan. The invention advantageously employs at least partially deacetylated chitin, chitosan, the properties of the polymer are advantageously further modified by linking aliphatic chains to at least some of the free amino groups of chitosan. Preferably, said aliphatic chains are linked via reacting aliphatic aldehydes with said amino groups. In a preferred embodiment of the invention, a system comprising N-imino derivative (Schiff base) of the chitosan is employed. In one aspect, the Schiff base is converted to an amine, for example by reducing with borohydride, cyanoborohydride or pycolineborohydride, thereby obtaining a stably alkylated chitosan, non-sensitive to acidity changes. In another aspect, the Schiff base-comprising chitosan is employed for agent delivery, while utilizing the sensitivity of the N-imine bond to the ambient acidity for eventual detachment of the hydrophobic chain and eventual release of the agent to the environment. The advantageous properties of the system have verified with model agents covering the property range of many beneficial active agents such as very hydrophobic and very hydrophilic compounds.

Actually, the field lacks delivery systems that are capable to (a) encapsulate both hydrophilic and hydrophobic active agents, (b) deliver them in both hydrophilic and hydrophobic environments, and (c) transfer active agents from one phase to another. The systems according to the invention have these properties and, in fact, represent omniphilic (likes everything) transporters. The instant systems are based on polysaccharides, which are available, safe, usually hypoallergenic, and their modifications can be tuned according to the specific requirements.

The invention will be further described and illustrated by the following examples.

### EXAMPLES

### Materials

Chitosan was purchased from Molekula (Munich, Germany). Propanal, butanal, octanal, decanal and coumarin were purchased from Acros Organics (Geel, Belgium). Hexanal, dodecanal, acetic acid, pyrene and potassium bromide FTIR grade were purchased from Sigma Aldrich (Steinheim, Germany). Rhodamine B (RB) was purchased from Fisher Chemicals (Loughborough, UK). Curcumin was purchased from Sigma-Aldrich Co. Pyrene was purchased from Sigma-Aldrich Co. Double deionized water (DDW) was obtained by filtering through a Treion TS1173 column. Deuterated solvents for NMR analysis (D20, AcOH-d4) were purchased from Armar Chemicals (Dottingen, Switzerland). Gas chromatography gases (helium, compressed air, nitrogen and hydrogen) were purchased from Gordon Gas and Chemicals (Tel Aviv, Israel) at a 99.999% grade. All regents, solvents and gases were used without further purification.

### Methods

Synthetic Procedure to Prepare Modified Amphiphilic Chitosan-Based Aggregates (CS-OPNs and CSreduced-OPNs)

In order to obtain dynamic (detachable)N-imine dynamic covalent bond (CS-OPNs), 1.5% w/w chitosan was dissolved in 100 mL of 0.6% w/w acetic acid at 23° C. Once the solution achieved sufficient homogeneity (60 min), an aldehyde was added and the solution was stirred overnight. The aldehydes are added in accordance with the required substitution degree, usually in excess. In this particular example, the ratio of 2.85 equivalent of aldehyde to one equivalent of chitosan's free amino groups was employed and resulted in dynamic (detachable)N-imine dynamic covalent bond. The amount of chitosan's free amino groups was determined according to the chitosan deacetylation degree, % DD.

In order to obtain a stable (non-dynamic) amine covalent bond the modified polymers (CSreduced-OPNs) were prepared as follows: 1.5% w/w chitosan was dissolved in 100 mL of 0.6% w/w acetic acid at 23° C. Once the solution achieved sufficient homogeneity (60 min), an aldehyde was added and the solution was stirred overnight. The aldehydes are added in accordance with the required substitution degree. In this particular example the ratio of 1.42 equivalent of aldehyde to one equivalent of chitosan's free amino groups was employed (the amount of chitosan's free amino groups was determined according to the chitosan deacetylation degree, % DD). Afterwards, 1.5 mL of a 10% wt sodium borohydride solution was added, resulting in reduction of the “dynamic” imine bond to a “non-dynamic” amine covalent bond, and the solution was stirred for an additional 2 hr at ambient temperature. The solution's pH was then adjusted to 14 by adding sodium hydroxide and the received precipitate was collected through 3 cycles of centrifugation and washed with water. The final precipitates were dried overnight under vacuum.

Degree of deacetylation (% DD) was determined by the first derivative UV method. Briefly, glucosamine and N-acetyl-glucosamine were separately dissolved at concentrated phosphoric acid in different concentrations. Their UV first derivative was then measured at 203 nm, indicating their respective weight fraction in a random commercial chitosan polymer. Next, chitosan samples were weighed in triplicate and heated to 60° C. for 40 min in 20 mL of 85% phosphoric acid. Samples were then diluted and heated for an additional 2 h in 60° C. The first derivative UV method was then applied again and compared to the original monomers' results, based on the formula %: DA=(100×m1/203.21)/((m1/203.21)+(m2/161.17)), where m1 is the mass of N-acetyl-glucosamine in a single chitosan sample, m2 is the mass of glucosamine in a single chitosan sample, and DA is the degree of acetylation. % DD=(100−% DA), found to be 94%. The value of % DD was also determined using 1H NMR scans by calculating its N-acetyl's integral value.

Synthetic Procedure to Prepare Modified Amphiphilic Carboxymethyl Cellulose (CMC)-Based Aggregates (CMC-OPNs)

CMC-based derivatives were synthesized by reaction with amines having four, six, eight or twelve carbon alkyl chain in the presence of EDC-NHS (1-Ethyl-3-(3-dimethylaminopropyl)carbodiimide-N-Hydroxysuccinimide) in water at room temperature. Following the preparation, the final products, CMC-4, 6, 8, or 12-amide were observed.

FTIR Spectra

FTIR spectra were recorded between 400 and 4000 cm⁻¹ by averaging 100 scans with a 4 cm⁻¹ resolution (Bruker Tensor 27 FTIR Spectrometer) using FTIR grade potassium bromide discs.

Spectrofluorometry

Modified biopolymer micellization was studied using pyrene as a fluorescent probe. Its fluorescent emission spectrum comprises of vibronic peaks that show a strong dependency on the solvent's polarity. The ratio between two specific peaks (i.e. I₃^{˜}383 nm and I₁^{˜}373 nm) in pyrene's spectrum were used as a quantitative measurement for its microenvironment's polarity. Any change in the surrounding polarity, such as when Pyrene is encapsulated from an aqueous environment by a hydrophobic-cored micelle, and is expressed by this ratio's value. Pyrene's highly hydrophobic nature and low water solubility (2-3 μM) ensure its preference to be present in hydrophobic over hydrophilic environments. These characteristics make Pyrene to be an ideal fluorescent probe for analyzing a substance's critical micelle concentration. Pyrene was first dissolved in absolute ethanol to obtain a 1 mM stock solution and then further diluted with various water-based modified biopolymer solutions at varying concentrations, always yielding a final concentration of 1 μM. Fluorescence spectra of modified biopolymer samples were measured in a standard 1 cm quartz cell using a computer-controlled Shimadzu, RF-5301PC spectrofluorometer equipped with a 150 W Xenon lamp (Ushio Inc., Japan). The excitation wavelength for pyrene was 330 nm with a slit width of 3 nm unless otherwise mentioned, and the emission band recorded was 360-380 nm with a slit width of 3 nm, at increments of 0.5 nm. All samples measured were kept at 23±1° C. All samples were made in duplicates and each duplicate was scanned twice. Spectra were not corrected for instrumental bias and may differ slightly in position and intensity from spectra collected on other instruments, however, any deviations from true spectra due to our instrumental configuration are consistent across all samples within this dataset. CAC values were calculated as the intersection between two linear lines depicting aggregate formation dependent on concentration in solution.

Rhodamine B Encapsulation Analysis

The Rhodamine B fluorescent probe was used in this study in order to demonstrate encapsulation within aggregates formed from the amphiphilic modified polysaccharides according to the invention. Rhodamine B were used at a final concentration of 10 μM in amphiphilic modified polysaccharides solutions with concentrations at least 3.5 times higher than their respective CACs. The excitation wavelengths for Rhodamine B was 557 nm with a slit width of 1.5 nm. Its emission bands was recorded at 590-610 nm with a slit width of 1.5 nm. All spectra were obtained at increments of 0.5 nm. All samples measured were kept at 23±1° C.

Rhodamine B Solubilization in Xylene

0.5 mL of aqueous solution of amphiphilic modified polysaccharides CS, CS-4, CS-8, CS-12 at concentrations at least 3.5 times higher than their respective CACs were added to 10 mL of a hydrophobic polar aprotic solvent (xylene) and the resulting solution was heated to 105° C. for 20 min in order to evaporate trace waters that were inserted with the aggregates. The solution had cooled spontaneously to room temperature and 3 mg of Rhodamine B were then added as a hydrophilic probe. Fluorescence spectra were measured in a standard 1 cm quartz cell using a computer-controlled Shimadzu, RF-5301PC spectrofluorometer equipped with a 150 W Xenon lamp (Ushio Inc., Japan). The excitation wavelength for Rhodamine B was 557 nm with a slit width of 1.5 nm, and the emission band recorded was 500-650 nm with a slit width of 1.5 nm, at increments of 1 nm.

UV-Vis Spectroscopy

Absorption measurements were recorded between 200 and 800 nm on a Shimadzu 1800 UV/Vis Spectrophotometer.

Confocal Microscopy

Laser scanning confocal microscopy (LSCM) imaging was performed using a Leica SP8 inverted confocal microscope (Germany) with an LASX software and an HC PL APO CS2 63×/1.20 water immersion objective. Pyrene was excited at 405 nm and emission was collected from 415 to 530 nm. Rhodamine B was excited at 552 nm and emission was collected from 565 to 650 nm. Fluorescent dye solutions were freshly prepared the day of imaging (10 μM), and were also inspected separately under the microscope to ensure no contaminants were present to obtain false micro particles.

Franz Diffusion Cell Model

The ability of the OPNs to enhance RB permeation in vitro was evaluated using a vertical static Franz diffusion cell apparatus (PermeGear, Inc., Hellertown, Pa., USA). The lipid phase was loaded with 250 μL of the sunflower oil-based OPNs containing RB and mounted on an artificial membrane that mimics a skin-like barrier (Strat-M® Membrane, Merck Millipore, Darmstadt, Germany). The receptor chamber was filled with 5 mL of phosphate buffered saline (pH 7.4), constantly stirred (500 rpm) and maintained at 32±0.5° C. Samples (250 μL) were withdrawn from the receptor aqueous phase at six predetermined time intervals (0.5, 1, 2, 4, 8 and 16 h), and the chamber was refilled with an equivalent amount of fresh buffer solution. The rate of RB permeation was determined fluorescently (excitation at 557 nm and emission at 600 nm) using a Fluoroskan Ascent Microplate reader (Thermo Scientific). All samples were tested in triplicate repetitions.

### Example 1

### OPNs Properties Determination

Some properties of the prepared modified polysaccharides are presented in Table 1, including critical aggregate concentration (CAC) via spectrofluorometric measurement, and characteristics of chitosan-based films, including thickness, water vapor transmission rate (WVTR), Young's modulus (YM), elongation at break (EB), and tensile stress (TS). The incorporation of hydrophobic substituents was reflected in the water vapor transmission rates. Since hydrophobic moieties repel water vapors from diffusing freely through the polymer chains, a reduction in WVTR values for the modified polymers as compared to the original chitosan (CS) was observed. Concerning mechanical properties such as roughness, rigidity, and elasticity, N-modification had, for the most part, an increasingly detrimental effect on the polymer integrity with increasing chain length, probably caused by a more significant separation between the polymer chains.

TABLE 1
Properties of chitosans hydrophobized by alkyl chains
linked to the free amino groups via Schiff base bond

| | CAC | WVTR | Thickness | YM | EB | TS |
|---|---|---|---|---|---|---|
| Polymer | [mg/mL] | [gr/hm²] | [μm] | [MPa] | [%] | [MPa] |
| CS | 0.59 ± 0.07 | 13.11 ± 0.67 | 21.3 ± 0.4 | 2311.7 ± 284.9 | 13.1 ± 2.5 | 73.7 ± 22.0 |
| CS-3 | 0.31 ± 0.02 | 12.52 ± 0.25 | 24.5 ± 0.8 | 1209.8 ± 303.5 | 9.9 ± 2.1 | 55.8 ± 17.6 |
| CS-4 | 0.21 ± 0.01 | 12.23 ± 0.25 | 25.6 ± 0.7 | 1387.3 ± 280.4 | 8.3 ± 0.8 | 47.4 ± 2.0 |
| CS-6 | 0.11 ± 0.01 | 11.71 ± 0.31 | 25.2 ± 3.7 | 1211.3 ± 215.3 | 5.2 ± 0.7 | 33.2 ± 10.0 |
| CS-6′ | 5.70 × 10⁻³ ± 0.25 × 10⁻³ | 12.38 ± 0.74 | 30.6 ± 2.4 | 1430.9 ± 85.4 | 6.6 ± 2.3 | 67.2 ± 19.2 |
| CS-8 | 1.92 × 10⁻² ± 0.19 × 10⁻² | 11.05 ± 0.62 | 32.6 ± 0.5 | 1151.1 ± 131.1 | 8.2 ± 2.4 | 39.5 ± 9.3 |
| CS-10 | 1.81 × 10⁻² ± 0.22 × 10⁻² | 8.84 ± 1.25 | 33.7 ± 5.2 | 623.7 ± 118.2 | 4.7 ± 1.5 | 19.5 ± 5.3 |
| CS-11′ | 0.79 × 10⁻³ ± 0.03 × 10⁻³ | 10.61 ± 0.39 | 38.5 ± 1.3 | 713.2 ± 100.2 | 5.3 ± 1.1 | 27.4 ± 3.8 |
| CS-12 | 6.01 × 10⁻³ ± 0.14 × 10⁻³ | * | 65.3 ± 1.1 | 408.2 ± 75.5 | 3.4 ± 0.4 | 4.3 ± 0.8 |

* CS-12 was not investigated for WVTR due to its brittleness.

### Example 2

### Encapsulation and Delivery Abilities of OPNs in Water

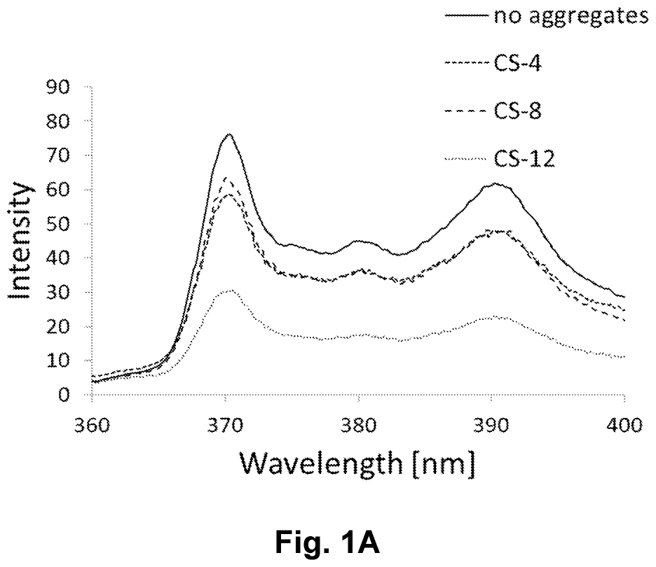

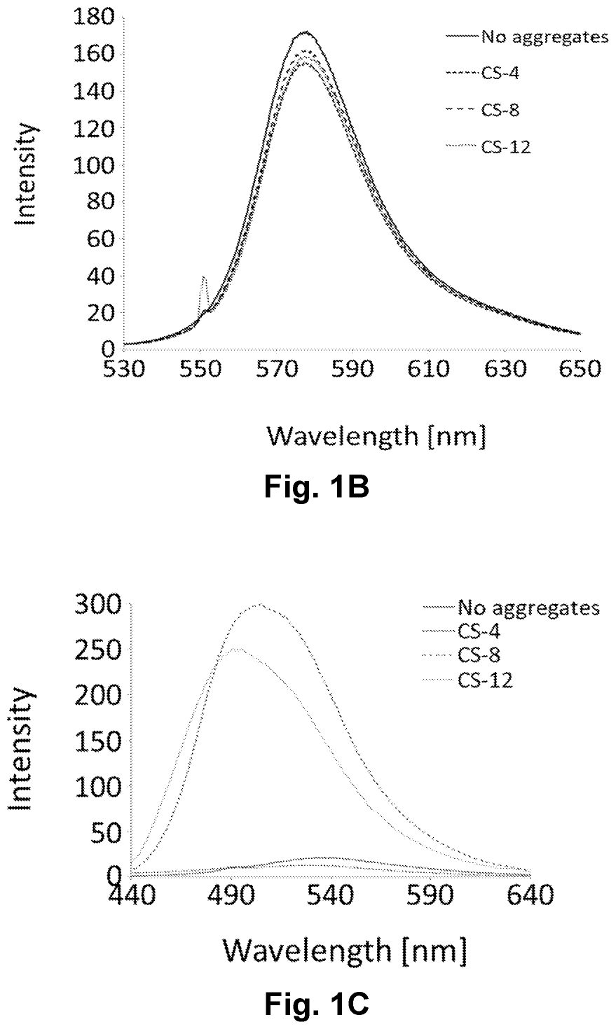

The delivery of Rhodamine B (RB) and pyrene as hydrophilic and hydrophobic fluorescent probes and curcumin as active hydrophobic agents was examined. FIG. 1 shows fluorescent spectra of various active agents encapsulated in the prepared aggregates.

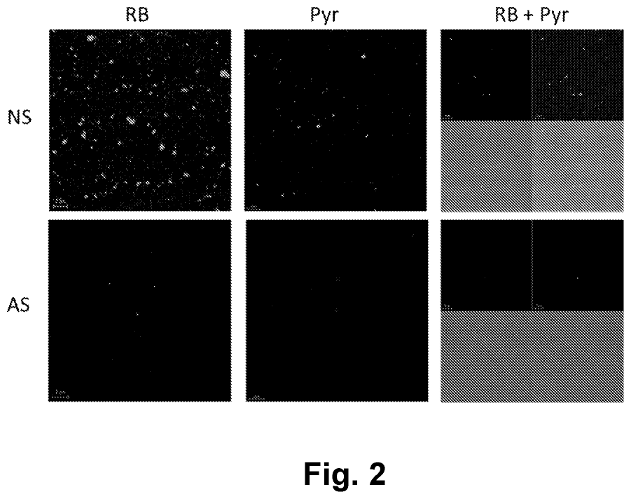

The encapsulation was imaged by CLSM (confocal laser scanning microscopy) for CS-4 and CS-12, the aggregates bearing the shortest and longest coupled side chains, respectively. The images showed the encapsulation of pyrene and Rhodamine B by essentially spherical sub-micron aggregates with a narrow size distribution. The aggregates displayed Brownian motion, indicating thermodynamic stability with no apparent agglomeration. Dye molecule encapsulation was imaged by CLSM, and corresponding images have shown a successful encapsulation of either probe, as ordered sphere-shaped sub-micron aggregates with good monodispersity. The formed vesicles, also called here omniphilic polysaccharide nanopackets (OPN) were seen to display Brownian motion in solution, indicating thermodynamic stability with no apparent agglomeration. Following their ability to individually encapsulate either pyrene or RB, OPNs were introduced to both of them together. CLSM images have revealed that the two probes' fluorescent signals all emanate from the same points of origin, proving that the prepared aggregates can simultaneously house hydrophilic and hydrophobic probes (FIG. 2) The prepared aggregates thus demonstrate an advantageous omniphilicity with a dual-housing capability for diverse guest types, either individually or simultaneously.

### Example 3

### Stimuli Responsiveness of OPNs

The N-imine dynamic covalent bond, linking between the chitosan (CS) backbone and the coupled aldehydes, was cleaved as a response to an external pH stimulus. This possibility adds to the designed systems a dynamic potential. The cleavage process was observed to proceed to the complete disassembly of the aggregates, taking place nearly instantly, taking less than 60 seconds, regardless the payload type or side chain length. Following the acidification, the aggregates ceased to be visible in the microscope. Control images with the same concentration of either fluorescent probe yielded identical images in which no discernible dye was visible (data not shown). Aggregates with two different guests have also shown susceptibility to N-imine cleavage upon triggering a similar pH stimulus, allowing the release of two different payloads at the same time (FIG. 2). The cleavage process reverted the polymer to a non-aggregated state, releasing the coupled aldehydes in addition to the payload.

### Example 4

### Abilities of OPNs to Perform Delivery in Hydrophobic Solvents and Phase Transport

The amphiphilic aggregates aqueous solutions were individually added to xylene, an organic hydrophobic solvent, and the mixtures were heated to remove the water fraction, leaving the aggregates in the organic phase. DLS scans evidenced the presence of self-assembled aggregates in this new microenvironment. Their CACs in xylene were found to demonstrate an opposite trend to that which had been found in the original aqueous solutions, as aggregates with longer side chains were of higher CACs (0.48, 0.09, and 0.02 mg/mL for CS-12, 8, and 4, respectively). It is therefore plausible that hydrophilic biopolymers with shorter side chains have a stronger driving force to aggregate in hydrophobic solvents.

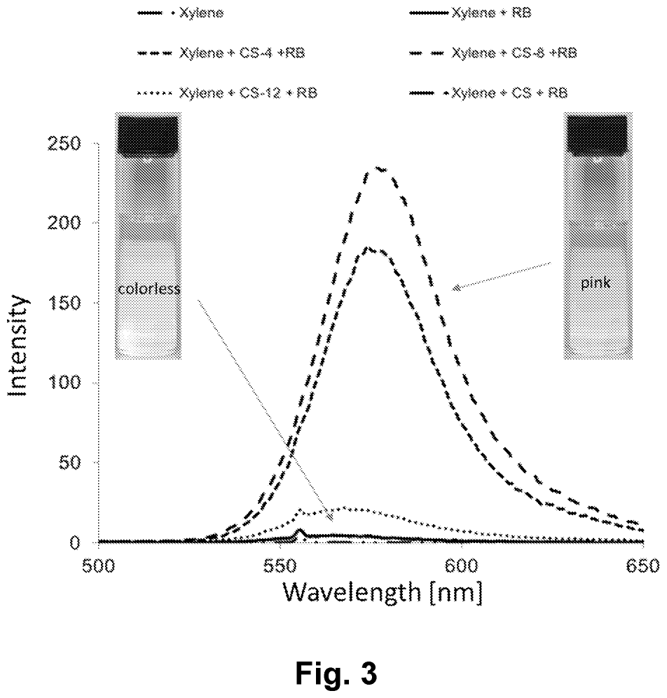

The introduction of hydrophilic RB, which is insoluble in xylene, was examined next. Spectrofluorometric scans and the appearance of a stable pink-violet color have shown RB solubility in the presence of aggregates. Additional scans have shown that RB encapsulation by CS-4 or CS-8 had a more significant effect on its fluorescent spectra than CS-12 did (FIG. 3).

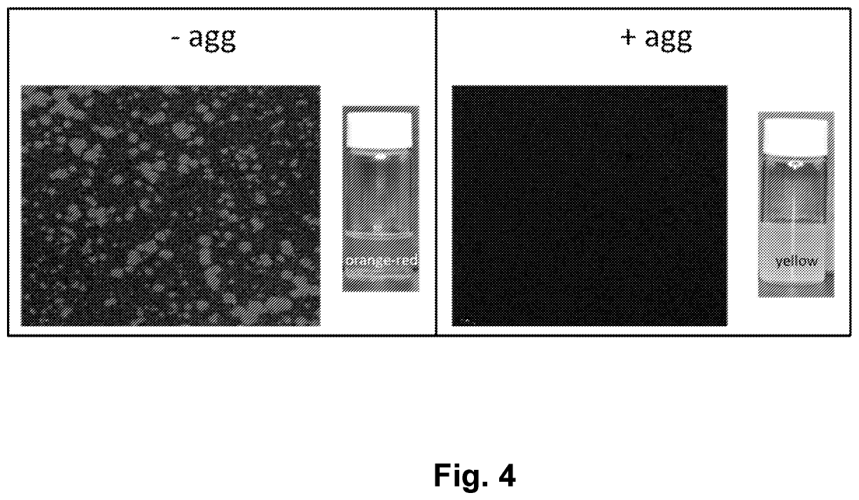

In a follow-up experiment, sunflower oil, widely used in food and cosmetics and comprised of fatty acids that resemble a physiological lipid environment, was used as a lipophilic medium. When added alone, no evidence of RB's solubility was observed until aggregates were added, directly necessitating them for its successful encapsulation (FIG. 4).

Aggregates loaded with RB have demonstrated long-term stability under lipophilic conditions, as was displayed with a stable color and via CLSM images. Notably, unlike the previously reported results in aqueous media, by introducing an acidic stimulus under lipid conditions no N-imine cleavage was achieved neither with a protic glacial acetic acid nor with AlCl₃ as a Lewis acid. Remarkably, prepared aggregates have shown the ability to transport active agents across different phase barriers without losing their overall structural identity or pre-encapsulated payloads. Consequently, the designed OPNs can endure a stable delivery of multiple active payloads through lipophilic media, later to be released when nearing their target. Being constructed of complex macromolecules, the OPNs can perform numerous structural self-adjustments simultaneously and provide different favorable microenvironments for various interactions at the same time. Such an advanced feature allows them to deal with miscellaneous combinations of various active molecules as well as bulk media, enabling cross-phase transportation capabilities.

### Example 5

### Application of OPNs in Food Storage

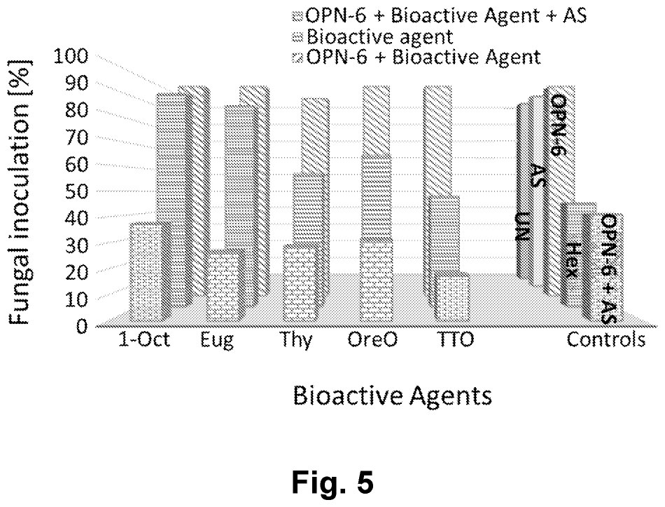

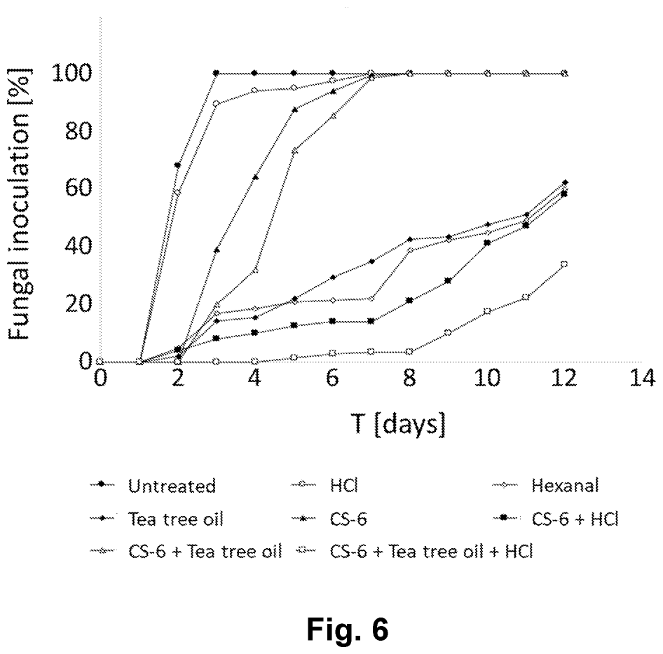

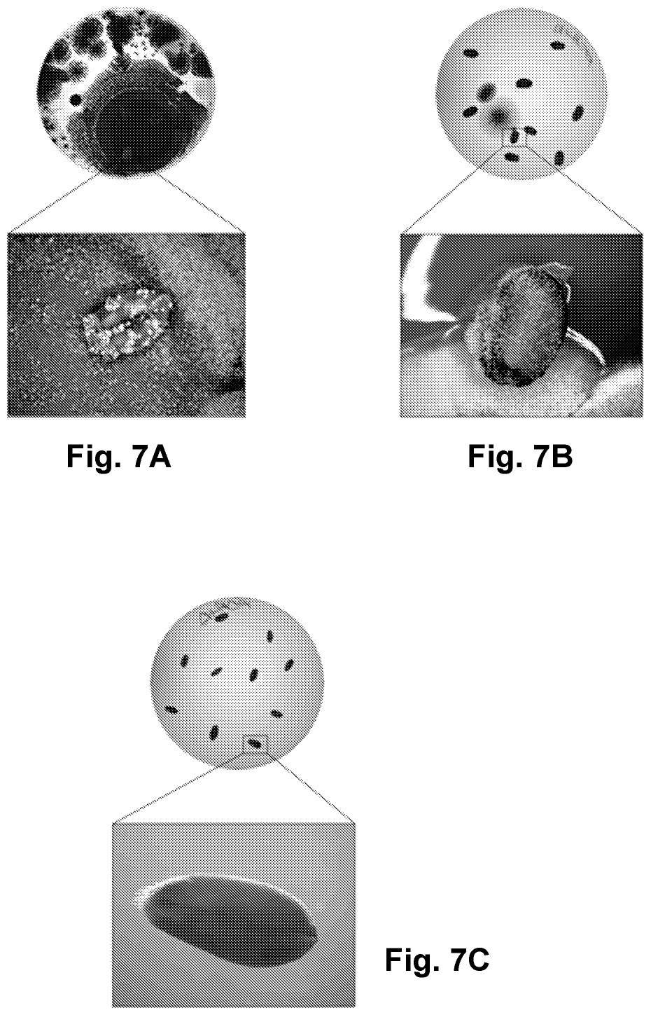

In order to assess the applicative potential of the encapsulating vesicles, they were tested on a wheat grains model. Modified chitosan CS-6 was prepared by coupling CS with hexanal, known as exhibiting antifungal activity. Various antifungal components were then encapsulated within CS-6, including 1-octanol, eugenol, thymol, oreganum oil, or tea tree oil, yielding five different formulations. Wheat grains were separately exposed to these different formulations that had been activated by an acidic stimulus. Exposure time lasted 20 days, and the grains were then placed on potato dextrose agar plates to promote microbial growth. Several control treatments were used and included acid-free inactivated formulations, antimicrobial agents that had been directly added, hexanal as the attached appendage in CS-6, and the used acidic stimulus. It was found that CS-6 significantly amplified the antimicrobial activity of the encapsulated active agent, regardless the agent type. The best results in fungal expression inhibition were obtained for acidic stimuli-activated aggregates. The treatments were most efficient when encapsulated agents were applied with acid, enabling releasing hexanal as an auxiliary antifungal agent that laid dormant until properly triggered (FIG. 5). The encapsulated tea-tree oil formulation, for instance, succeeded in almost fully inhibiting fungal growth on wheat grains for up to 8 days, whereas the fungal contamination took 12 days to grow to 40% of the maximal level (FIG. 6). A formulation that had not been exposed to the necessary external pH trigger reached said 40% level as early as within 4 days.

### Example 6

### Application of OPNs in Agriculture

The encapsulating vesicles were further tested by application on plants. Specifically, CMC-OPNs and CSred-OPNs were utilized to encapsulate and deliver plant nutrients.

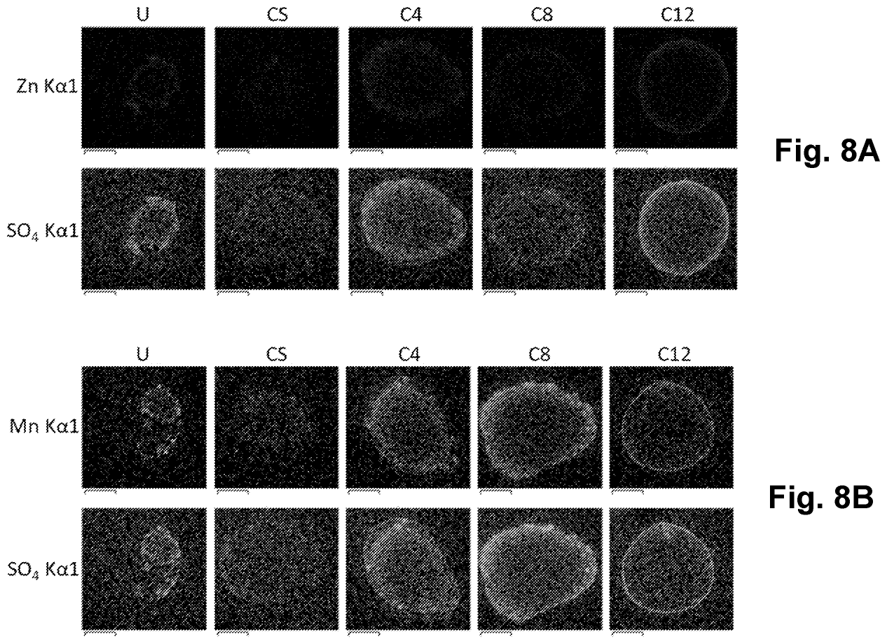

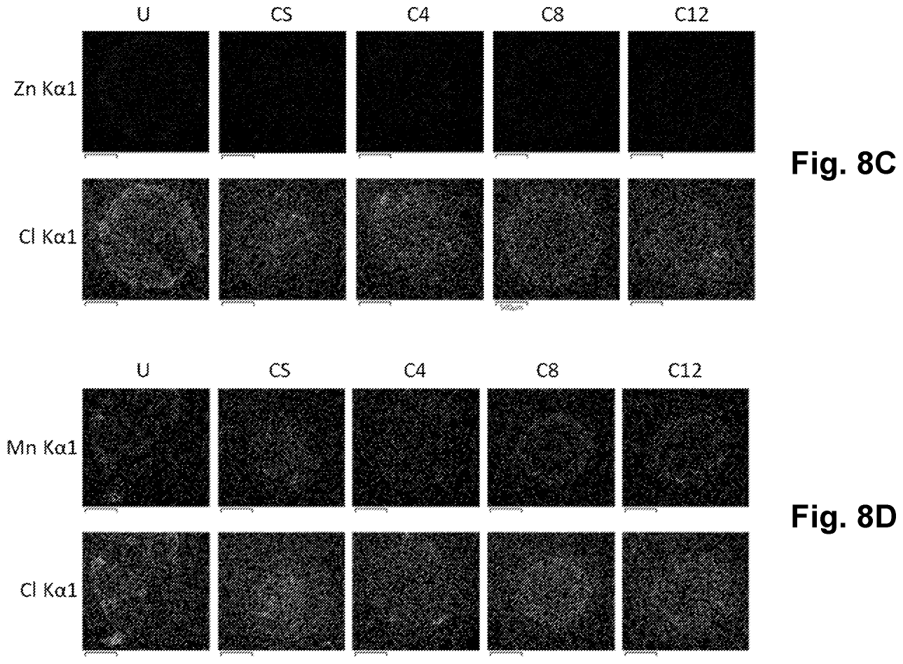

In one experiment, 20 ml of aqueous solution containing 100 ppm of ZnSO₄ and MnSO₄ microelements encapsulated in 2.5 mg/ml of CSreduced-OPNs were applied on pepper leaves. As can be seen in SEM-EDS (Scanning Electron Microscopy with Energy Dispersive Spectroscopy) microscopic images that monitored Zn, Mn and SO₄ ions in the plant leaves, the presence of OPNs resulted in significant enhancement of the microelement amount (FIG. 8).

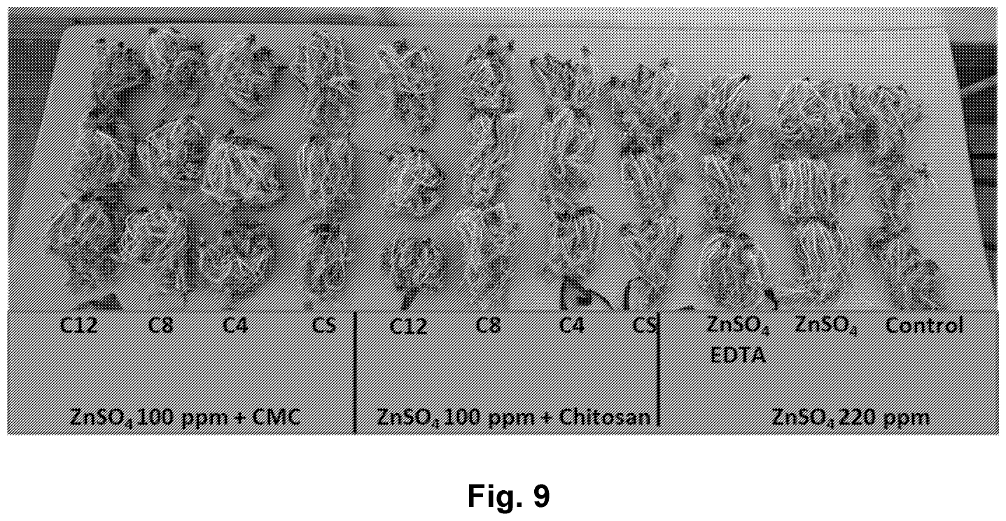

In another experiment, 100 ppm of ZnSO₄ were added to 20 ml of aqueous solution containing 1.25 mg of CMC-OPNs or CSreduced-OPNs. The solution was applied on Maize Plant (that suffer from Zn deficiency). Plant quality parameters were measured after 14 days. The treatment with aqueous solution containing 200 ppm of ZnSO₄ enhanced by EDTA chelate was used as a positive control. The roots of the treated plants after 14 days are shown in FIG. 9. As can be seen, although the positive control treatment contained twice of the Zn amount in comparison to OPN-treatments, the roots of the plants that were treated with ZnSO₄ encapsulated in OPNs were larger.

### Example 7

### Application of OPNs in Medicine for Transdermal Delivery

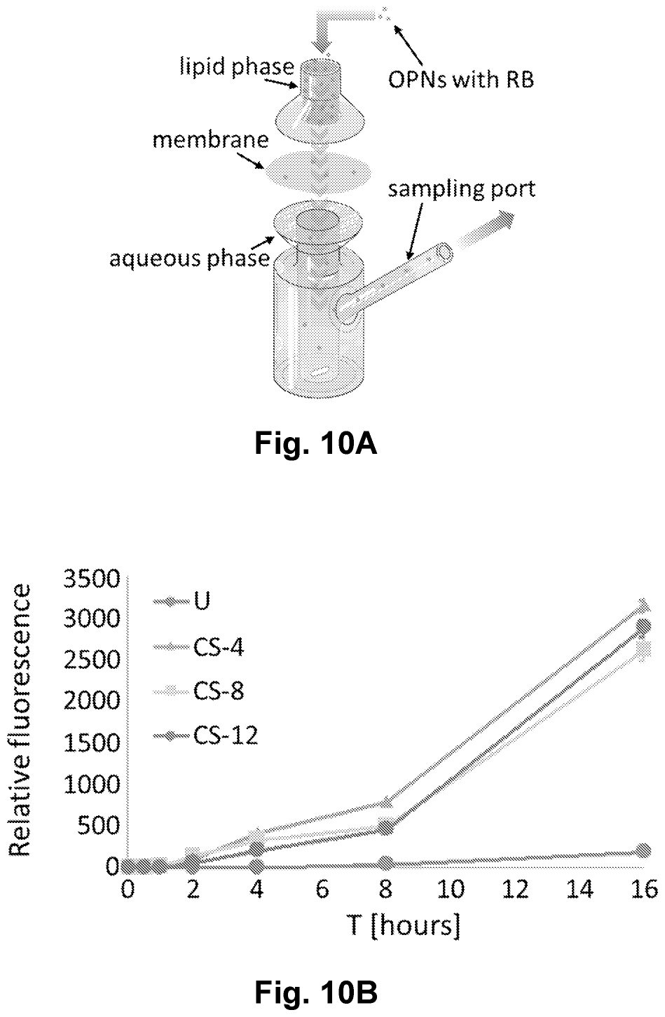

The OPNs' phase transfer ability was demonstrated using a Franz diffusion cell, which is a common in vitro model used to estimate the ability of compounds to be transferred across lipid barriers into an aqueous phase. This model is widely used in pharmacology and cosmetics for transdermal delivery studies, and can even be used to study cuticle penetration in plant leaves. In this experiment, RB containing OPNs were dissolved in sunflower oil and allowed to permeate spontaneously across an artificial skin-like lipophilic membrane onto an aqueous solution, where their fluorescent intensity was sampled. The results show all of the inspected OPNs have successfully transported encapsulated RB across the lipophilic barrier. As a control test, it can be seen that without OPNs, RB (red) scarcely permeates across the membrane (FIG. 10).

While the invention has been described using some specific examples, many modifications and variations are possible. It is therefore understood that the invention is not intended to be limited in any way, other than by the scope of the appended claims.

1. An amphiphilic aggregate comprising an ionic polysaccharide modified by covalently linked moieties that contain aliphatic chains comprising between 2 and 26 carbon atoms and degree of substitution with aliphatic moieties constitutes between 0.5 and 70%, said modified polymer exhibiting a critical aggregate concentration (CAC) of between 0.00005 and 2 mg/ml.

2. The aggregate of claim 1, wherein said moiety is linked via a bond selected from the group consisting of amine bond, amide bond, N-imine bond, ester bond, and ether bond.

3. The aggregate of claim 1, wherein said modified polysaccharide complies with the Rhodamine test.

4. A delivery system comprising an amphiphilic aggregate essentially consisting of a modified polysaccharide complying with the Rhodamine test.

5. The delivery system of claim 4, wherein said polysaccharide is modified by covalently linked moieties that contain aliphatic chains comprising between 2 and 26 carbon atoms linked via a bond selected from the group consisting of amine bond, amide bond, N-imine bond, ester bond, and ether bond.

6. The delivery system of claim 5, wherein said degree of substitution with aliphatic moieties constitutes between 0.5 and 70%, said modified polymer exhibiting a critical aggregate concentration (CAC) of between 0.00005 and 2 mg/ml.

7. The delivery system of claim 4, configured for delivering hydrophobic and hydrophilic agents into or between hydrophilic and lipophilic environments.
